(19) United States
(12) Patent Application Publication
(10) Pub. No.: US 2019/0328785 A1
(43) Pub. Date: Oct. 31, 2019

(54) FLT3-BINDING CHIMERIC ANTIGEN RECEPTORS, CELLS, AND USES THEREOF

(71) Applicant: The Board of Regents of the University of Oklahoma, Norman, OK (US)

(72) Inventors: Zhizhuang Zhao, Edmond, OK (US); Suraj Pratap, Oklahoma City, OK (US)

(21) Appl. No.: 16/399,052

(22) Filed: Apr. 30, 2019

Related U.S. Application Data
(60) Provisional application No. 62/664,629, filed on Apr. 30, 2018.
(60) Provisional application No. 62/741,224, filed on Oct. 4, 2018.

(51) Int. Cl. A61K 35/17; A61P 35/02; A61K 9/00; C07K 14/705

(52) U.S. Cl. CPC A61K 35/17; A61P 35/02; C07K 14/70521; A61K 9/0017; A61K 9/0053; A61K 9/0073

(57) ABSTRACT

A chimeric antigen receptor (CAR) comprising (1) an extracellular portion of Fms-related tyrosine kinase 3 ligand (FLT3L) that binds to Fms-related tyrosine kinase 3 (FLT3), (2) a transmembrane domain, (3) a costimulatory signaling domain, and (4) an intracellular signaling domain. A nucleic acid sequence encoding the CAR. A vector and cell comprising the nucleic acid sequence encoding the CAR. A cell expressing the CAR. A composition of cells expressing the CAR. A method of administering the composition of cells expressing the CAR to a subject for stimulating in the subject an immune response against cells which express FLT3.

### CROSS REFERENCE TO RELATED APPLICATIONS/INCORPORATION BY REFERENCE STATEMENT

The present patent application claims priority to U.S. Provisional Patent Application Ser. No. 62/664,629, filed on Apr. 30, 2018, and to U.S. Provisional Patent Application Ser. No.62/741,224, filed on Oct. 4, 2018, the entire contents of each of which is hereby expressly incorporated herein by reference.

### BACKGROUND

Current therapies for acute myeloid leukemia (AML) and acute lymphocytic (lymphoblastic) leukemia (ALL) use varying combinations of intensive chemotherapeutic drugs given repetitively in the form of multiple cycles over many months. Conventional chemotherapeutic agents cause numerous side effects that can lead to short-term and long-term morbidity. Numerous studies have found that between 50-70% patients of AML patients and up to 30% of ALL patients relapse within five years of achieving remission despite receiving highly toxic multi-agent chemotherapy. It is clear from the survival statistics that current therapies for leukemia need significant improvement.

In the past few years, immunotherapy has begun to drastically change the landscape of cancer treatment. Deeper understanding of T-cell receptor (TCR) activation and cytotoxicity has finally enabled scientists to grow T-lymphocytes ex-vivo and modify their antigen receptors with great precision using chimeric antigen receptor (CAR) technology. Newer generation CAR T-cells have been further improved to provide both primary and secondary signals required for T-cell activation. For instance, numerous CARs utilizing novel co-stimulatory domains are being developed and tested, broadening our understanding of how the co-stimulation affects T-cell proliferation and longevity.

Healthy human T-cells express TCR, an antigen recognition receptor, which is composed of alpha and beta subunits that work in conjunction with co-stimulatory molecules and an intracellular signaling domain. The fundamental idea behind a CAR T-cell is to design a T-cell receptor that has affinity for a specific tumor associated antigen (TAA) thereby enabling the T-cell to target and destroy the particular cancer cell. The CAR is a modified TCR that comprises a purposefully designed antigen recognizing moiety fused in series with co-stimulatory and intracellular signaling molecules. The extracellular domain of CAR, also called the antigen binding domain, is responsible for tumor specificity and overall affinity. The transmembrane region is the segment that passes through the cell membrane and is frequently made up of the transmembrane region of proteins belonging to the cluster of differentiation (CD) family. The intracellular signaling domain is generally made up of proteins like the CD3 zeta chain that are responsible for downstream signaling from the CAR and can be supplemented by a variety of co-stimulatory proteins such as, CD28, 4-1BB (CD137), and OX40 (CD134).

CAR T-cell based therapies have been shown to be effective against ALL, but similar therapies against AML are still at an early stage. Many early CAR T-cell clinical trials (Anti-CD19, Ant-CD123 and Anti-HER-2 CAR T-cells) were closed due to therapy related deaths which has further highlighted the concerns about toxicity from genetically modified T-lymphocytes. As a result, identification of the appropriate target antigen is perhaps the most crucial determinant of the anti-tumor potency and the eventual clinical success of any CAR T-cell. Since the extra-cellular domains of the majority of CARs are derived from monoclonal antibodies generated in animals these CAR T-cells can cause toxicity to non-cancer cells (off-target toxicity) and also pose the risk of allergic (hypersensitivity) reactions. Both off-target toxicity and hypersensitivity reactions are major factors responsible for CAR T-cell related morbidity and mortality. Furthermore, there is a high risk of immunogenic clearance that can potentially reduce the anti-tumor potency of future doses. Recently researchers have been trying to overcome this challenge by creating CARs with partially or fully humanized antigen receptors. Most extra-cellular domains are made up of single-chain variable fragments (scFv) derived from monoclonal antibodies directed against specific TAAs. The ideal tumor antigens are those which are unique to cancer cells and are expressed on all (or almost all) cells of a particular type of cancer. A CAR T-cell therapy utilizing an antigen which is not present in sufficiently high proportion of cancer cells can cause reduction in cancer cells but may not be capable of eradicating the disease (inducing remission). Although the efficacy of CAR T-cells is not directly proportional to antigen-antibody affinity, the interaction should be strong enough to redirect the T cell cytotoxicity towards cancer cells. Some recent studies have suggested that CARs with moderate affinity towards TAA may have good anti-cancer cytotoxicity making them better candidates for clinical application.

Anti-CD19 CAR T-cell based therapies have been shown to be effective against ALL and lymphoblastic lymphoma but similar therapies against AML are still urgently needed. At present there are no FDA-approved adoptive T-cell based therapies for AML patients. Many recent clinical trials (JCAR015 Trial, UCART123 Trial) have highlighted the concerns about toxicity and therapy related deaths from anti-leukemia CAR T-cells.

### BRIEF DESCRIPTION OF THE DRAWINGS

Several embodiments of the present disclosure are hereby illustrated in the appended drawings. It is to be noted however, that the appended drawings only illustrate several embodiments and are therefore not intended to be considered limiting of the scope of the present disclosure. The patent or application file contains at least one drawing executed in color. Copies of this patent or patent application publication with color drawing(s) will be provided by the Office upon request and payment of the necessary fee.

FIG. 1 shows a DNA sequence (SEQ ID NO:1, also see Table 1) and corresponding protein sequence (SEQ ID NO:2, also see Table 2) of an exemplary, non-limiting, CAR construct of the present disclosure (referred to herein as FLCAR) which encodes a portion of an extracellular portion of Fms-related tyrosine kinase 3 ligand (FLT3 ligand, also referred to herein as “FL”), a transmembrane region of CD28, and a CD3-zeta chain intracellular signaling domain. Underlining highlights the signal sequence of FL and the transmembrane region of CD28. Four tyrosyl residues embedded in the immunoreceptor tyrosine-based activation motifs (ITAMs) of the TCR are indicated in bold-face. Bases 1-477 encode the extracellular region of FL used in the present construct. Bases 478-480 and 731-733 encode glycine spacers. Bases 481-730 (in boldface) encode a transmembrane and costimulatory portion of CD28. Bases 734-1059 (in Italic) encode the CD3-zeta intracellular signaling domain.

FIG. 2 shows a schematic structure of an FLCAR constructed in accordance with the present disclosure and its signaling in T-cells.

FIG. 3 shows expression of FLCAR in cells. (A) Expression of FLCAR in Jurkat cells. Jurkat cells were infected with recombinant lentivirus carrying the pHIV-EGFP vector or the FLCAR construct. After 4 days of infection, cells were extracted and separated on 12.5% SDS gel for western blot analysis to detect FLCAR expression by using antibodies against phospho-CD3-Zeta and CD3-Zeta. HRP-conjugated anti-GAPDH was used for loading control. Size of markers and positions of expected proteins are indicated. Note that FLCAR shows as a 41 kDa band that is very close the calculated molecular weight of 40,040. Phosphorylated FLCAR runs as heterogeneous bands with slightly higher molecular size than the non-phosphorylated form. (B) Expression of FLCAR in primary human T-cells. Primary cells were infected with recombinant lentivirus carrying the pHIV-EGFP vector or the FLCAR construct. After 4 days of infection, EGFP-positive cells were isolated by cell sorting and further cultured for 4 days. Cells were extracted and separated on 10% SDS gel for western blot analysis to detect FLCAR expression by using antibodies against phospho-CD3-Zeta and CD3-Zeta. HRP-conjugated anti-GAPDH was used for loading control. Positions of expected proteins are indicated. Note that phosphorylated FLCAR runs slightly higher than the non-phosphorylated form.

FIG. 4 shows IL-2 expression induced by co-incubation of effector (FLCAR T-cells) and target (FLT3 expressing cells) using real time PCR analysis. (1) Total RNAs were isolated from Jurkat-EGFP or Jurkat-FLCAR cells with or without pre-incubation (for 0 or 24 hr) with MV-4-11, HCD-57-JAK2V617F, and HCD-57-FLT3-ITD. (2) Single strand cDNAs were synthesized using the QuantiTect reverse transcription kit (Qiagen). (3) Real time PCR was performed with iQ SYBR Green Supermix (Bio-Rad) with PCR primers GGGACTTAATCAGCAATATCAACG (SEQ ID NO: 24) and CTACAATGGTTGCTGTCTCATCAG (SEQ ID NO: 25) for IL-2, and AGGGCTGCTTTTAACTCTGGTAA (SEQ ID NO: 26) and TGGGTGGAATCATATTGGAACAT (SEQ ID NO: 27) for GAPDH. (4) The top figure shows the real time PCR profile of IL-2 and GAPDH in Jurkat-EGFP and Jurkat-FLCAR. The table below summarizes the CT (threshold cycle) values of samples from indicated cells or cell mixtures. Note that Jurkat-EGFP control cells did not yield any IL-2 PCR product, while Jurkat-FLCAR cells produced clear IL-2 product that was further enhanced by co-culture with FLT3-ITD expressing MV-4-11 or mouse HCD-57-FLT3-ITD cells as represented by smaller ΔCT values.

FIG. 5 shows cytotoxicity against FLT3 expressing MV-4-11 leukemia cells upon co-incubation with FLCAR T-cells. Equal numbers of MV-4-11 cells were co-cultured at indicated ratios with Jurkat cells expressing control EGFP or FLCAR for 48 hr. Cells were stained with anti-CD33 and 7AAD before flow cytometric counting analysis. Viable MV-4-11 cells are CD33⁺/7AAD⁻, while viable Jurkat cells are CD33⁻/7AAD⁻. Error bars denote standard deviation (n=3).

FIG. 6 shows that administration of FLCAR T-cells prolongs survival of mice engrafted with leukemia cells. MV-4-11 cells (1×10⁶) were mixed with 1×10⁶ primary T-cells expressing the EGFP control or FLCAR and then engrafted through retro-orbital injection in 15 week-old immuno-deficient NSG-SGM mice (3 each, all female). Data show mouse survival curve.

FIG. 7 shows that administration of FLCAR T-cells prolongs survival of mice engrafted with leukemia cells. MV-4-11 cells (1×10⁶) were injected retro-orbitally into 8 week-old immuno- deficient NSG-SGM mice (female, 5 in each group) on day one. On day 2 and day 4, 1×10⁶ primary T-cells expressing EGFP control or FLCAR were then introduced through retro-orbital injection. Data show mouse survival curve. P values are based on Log-rank test.

FIG. 8 shows that administration of FLCAR-T cells prolongs survival of patient-derived xenograft (PDX) mice. Primary leukemia cells from a FLT3-ITD-positive AML patients (5×10⁶, >80% blast cells) were injected retro-orbitally into 8 week-old immuno-deficient NSG-SGM mice (male, 5 in each group) on day one. On day 3 and day 5, 1.5×10⁶ primary T-cells expressing EGFP control or FLCAR were then introduced through retro-orbital injection. Data show mouse survival curve. P=0.02 (based on Log-rank test).

### DETAILED DESCRIPTION

Fms-related tyrosine kinase 3 ligand (FLT3L, or FL) is an endogenous protein which functions as a hematopoietic growth factor by binding and activating Fms-related tyrosine kinase 3 (FLT3), a receptor found on a variety of hematopoietic cells. The human FL gene encodes a 235-amino acid type I transmembrane protein comprising four domains including, an N-terminal 26-residue signal peptide, a 156-residue extracellular domain, a 23-amino acid transmembrane domain, and a 30-residue cytoplasmic domain. Soluble FL is thought to be released into circulation from the cell membrane by protease cleavage. It is a noncovalently-linked dimer and contains 6 cysteine residues that form intramolecular disulfides.

The present disclosure is directed to cells, compositions, vectors, and methods for redirecting the affinity and cytotoxicity of lymphocytes (e.g., T-lymphocytes) towards leukemic and non-leukemic myeloid cells by artificially expressing FLCAR, a surface-exposed chimeric antigen receptor (CAR) that contains an FLT3-binding portion of human FL at its extracellular domain. The cells expressing the FLCAR can be used in therapies for treating conditions associated with cells which express FLT3 such as, but not limited to, acute myeloid leukemia (AML), myelodysplastic syndromes (MDS), and patients of chronic myeloid leukemia (CML) in blast crisis, and as a conditioning agent to achieve myeloablation during hematopoietic stem cell transplantation (HSCT). HSCT is used to treat many hematological and non-hematological cancers as well as some non-cancerous diseases. The FLCAR comprises an FL extracellular region (portion), defined herein as a portion of the FL protein (or a variant thereof) which is able to bind with high affinity to the FLT3. The FLCAR may comprise, in series, (1) an FL extracellular region, (2) a transmembrane domain (e.g., comprising a transmembrane portion of CD28 protein), (3) one or more costimulatory domains (e.g., comprising a costimulatory portion of CD28 protein), and (4) an intracellular signaling domain (e.g., a CD3-zeta signaling domain). FLCAR as used herein refers to an FL extracellular region-containing CAR cell. In one non-limiting embodiment the FLCAR comprises an FL extracellular region, transmembrane and costimulatory juxtamembrane regions derived from CD28, and a CD3-zeta intracellular signaling domain comprising a plurality of ITAMs (usually four or more). When a DNA sequence encoding an FLCAR construct is introduced into a T-cell (e.g., by a lentiviral vector or other suitable vector) causing expression of the FLCAR, the resultant FLCAR T-cells are capable of selectively inhibiting FLT3-expressing leukemia cells, FLT3-expressing myeloid cells, and FLT3-expressing bone marrow progenitor cells.

FLT3 is expressed in many malignant myeloid cells, and its gain-of-function mutation is frequently found in acute myeloid leukemia (AML). The FLCAR T-cell immunotherapy described herein can be used in treatment of patients with acute myeloid leukemia (AML) and myelodysplastic syndromes (MDS), and patients of chronic myeloid leukemia (CML) in blast crisis, and can be used as a myeloablative treatment prior to hematopoietic stem cell transplantation (HSCT) procedures, for example in patients with certain non-cancerous diseases including but not limited to sickle cell anemia and metabolic disorders (such as Hurler's disease), who might benefit from HSCT.

Before describing various embodiments of the present disclosure in more detail by way of exemplary description, examples, and results, it is to be understood that the present disclosure is not limited in application to the details of methods, constructs, cells, and compositions as set forth in the following description. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that other embodiments of the inventive concepts may be practiced without these specific details. In other instances, features which are well known to persons of ordinary skill in the art have not been described in detail to avoid unnecessary complication of the description.

All patents, published patent applications, and non-patent publications referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the constructs, cells, compositions and methods used, or the variation that exists among the study objects. Further, in this detailed description and the appended claims, each numerical value (e.g., temperature or time) should be read once as modified by the term “about” (unless already expressly so modified), and then read again as not so modified unless otherwise indicated in context. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

Also, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc., all the way down to the number one (1). Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 1-20, 10-50, 50-100, 100-1,000, 1,000-3,000, 2,000-4,000, etc.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The natural amino acids, where designated as such herein, include and may be referred to herein by the following designations: alanine: ala or A; arginine: arg or R; asparagine: asn or N; aspartic acid: asp or D; cysteine: cys or C; glutamic acid: glu or E; glutamine: gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

For purposes of classifying amino acids substitutions as conservative or nonconservative, amino acids are grouped in one embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of exemplary conservative amino acid substitutions have been constructed and are known in the art. In certain embodiments herein which reference possible substitutions, examples of interchangeable amino acids include, but are not limited to the following: arginine and lysine; glutamate and aspartate; serine and threonine; glutamine and asparagine; and valine, leucine and isoleucine. In other embodiments, the following substitutions can be made: Ala (A) by leu, ile, or val; Arg (R) by gln, asn, or lys; Asn (N) by his, asp, lys, arg, or gln; Asp (D) by asn, or glu; Cys (C) by ala, or ser; Gln (Q) by glu, or asn; Glu (E) by gln, or asp; Gly (G) by ala; His (H)by asn, gln, lys,or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe, or tyr; Tyr (Y) by trp, phe, thr, or ser; and Val (V) by ile, leu, met, phe, or ala.

Other considerations for amino acid substitutions include whether or not the residue is located in the interior of a protein or is solvent- (i.e., externally) exposed. For interior residues, conservative substitutions include for example: Asp and Asn; Ser and Thr; Ser and Ala; Thr and Ala; Ala and Gly; Ile and Val; Val and Leu; Leu and Be; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Be and Val; and Phe and Tyr.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” a uracil “U” or a “C”). The term nucleobase also includes non-natural bases as described below. The term “nucleic acid” encompasses the terms “oligonucleotide” and “polynucleotide,” each as a subgenus of the term “nucleic acid.”

As used herein, the terms “complementary” or “complement” also refer to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

The term “homologous” or “% identity” as used herein means a nucleic acid (or fragment thereof), or a protein (or a fragment thereof) having a degree of homology to the corresponding natural reference nucleic acid, or protein, that is at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% identical thereto. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

Percentage sequence identities can be determined with protein sequences maximally aligned by the Kabat numbering convention. After alignment, if a particular polypeptide region is being compared with the same region of a reference polypepetide, the percentage sequence identity between the subject and reference polypeptide region is the number of positions occupied by the same amino acid in both the subject and reference polypeptide region divided by the total number of aligned positions of the two regions, with gaps not counted, multiplied by 100 to convert to percentage.

In one embodiment “% identity” represents the number of amino acids which are identical at corresponding positions in two sequences of a protein having the same or similar activity. For example, two amino acid sequences each having 100 residues will have at least 90% identity when 90 of the amino acids at corresponding positions are the same. Similarly, in one embodiment “% identity” represents the number of nucleotides which are identical at corresponding positions in two sequences of a nucleic acid encoding the same or similar polypeptides. For example, two nucleic acid sequences each having 100 nucleotides will have 90% identity when 90 of the nucleotides in homologous positions are the same.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Another algorithm is the WU-BLAST (Washington University BLAST) version 2.0 software (WU-BLAST version 2.0 executable programs for several UNIX platforms). This program is based on WU-BLAST version 1.4, which in tum is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3: 266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

As used herein, “hybridization,” “hybridizes” or “capable of hybridizing” is understood to mean the forming of a double or triple stranded molecule or a molecule with partial double or triple stranded nature. The term “anneal” as used herein is synonymous with “hybridize.” The term “hybridization,” “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Non-limiting applications include isolating a nucleic acid, such as a gene or a nucleic acid segment thereof, or detecting at least one specific mRNA transcript or a nucleic acid segment thereof, and the like. Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid, the length and nucleobase content of the target sequence, the charge composition of the nucleic acid, and to the presence or concentration of formamide, tetramethylammonium chloride or other solvent in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization to achieve varying degrees of selectivity of a nucleic acid towards a target sequence are used. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suit a particular application.

In certain embodiments herein, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

The term encoding” as used herein refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Reoviridae family. Lentiviruses are unique among the retroviruses in being able to infect nondividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A nucleic acid is “operably linked” when it is placed into a functional relationship with another nucleicacid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “bind,” “binds,” or “interacts with” means that one molecule recognizes and adheres to a particular second molecule in a sample or organism, but does not substantially recognize or adhere to other structurally unrelated molecules in the sample.

By the term “specifically binds,” as used herein with respect to an antibody, is meant an antibody which recognizes a specific antigen, but does not substantially recognize or bind other molecules in a sample.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which includes an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, et al. Examples of viral vectors include, but are not limited to, adenoviral vectors, adeno-associated virus vectors, and retroviral vectors. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by attenuating the HIV virulence genes, for example, the genes env, vif, vpr, vpu, and nef are deleted making the vector biologically safe. In other embodiments of the present disclosure, a gamma retrovirus may be used as the transfecting agent.

Where used herein the term “wild-type” refers to the typical form (genotype and/or phenotype) of a bacterium, gene, nucleic acid, protein, or peptide as it occurs in nature and/or is the most common form in a natural population. In reference to a gene or nucleic acid, the term “mutation” refers to a gene or nucleic acid comprising an alteration in the wild type, such as but not limited to, a nucleotide deletion, insertion, and/or substitution. A mutation in a gene or nucleic acid generally results in either inactivation, decrease in expression or activity, increase in expression or activity, or another altered property of the gene or nucleic acid. In reference to a protein, the term “mutation” refers to protein comprising an alteration in the wild type, such as but not limited to, one or more amino acid deletions, insertions, and/or substitutions. A mutation in a protein may result in either inactivation, a decrease in activity or effect (e.g., binding), or an increase in activity or effect (e.g., binding), or another altered property or effect of the protein.

Where used herein, the terms “costimulatory signaling region,” and “costimulatory signaling domain” refer to an intracellular sequence derived from a costimulatory molecule which functions in concert with an intracellular signaling region (domain) such as a CD3-zeta signaling region to mediate a co-stimulatory response by the T-cell, such as proliferation. The costimulatory signaling region (domain) produces a costimulatory signal, which in combination with a primary signal from the intracellular signaling region (domain) leads to T-cell proliferation and/or upregulation or down regulation of key molecules. Examples of costimulatory molecules from which the costimulatory signaling region (domain) is derived include, but are not limited to CD27, CD28, 4-1BB, OX40, CD30, CD40, PD-1, ICOS, lymphocyte function associated antigen-1 (LFA-1), CD2, CD7, LIGHT, NKG2C, B7-H3, and a ligand that specifically binds with CD83. Other costimulatory molecules and costimulatory signaling domains are shown in U.S. Pat. No. 9,914,909, the entirety of which is expressly incorporated herein by reference.

Turning again to the presently disclosed constructs, cells, compositions, vectors, and methods, an FLCAR comprising an extracellular FLT3-binding domain enables the FLCAR, when expressed on the surface of a T-cell, to direct T-cell activity to those cells expressing FLT3, a receptor expressed on the surface of certain tumor cells and healthy cells. The transmembrane portion of the FLCAR may comprise, in non-limiting embodiments, a CD4 transmembrane domain, a CD8 transmembrane domain, a CD28 transmembrane domain, a CD3 transmembrane domain, or a 4IBB transmembrane domain. The intracellular signaling domain includes the signaling domain from the zeta chain of the human CD3 complex (CD3-zeta chain) and one or more costimulatory signaling domains, such as but not limited to, a CD28, CD27, or 4-1BB costimulatory signaling domain. The inclusion of a costimulatory signaling domain, such as the CD28 costimulatory signaling domain in series with CD3-zeta chain in the intracellular region enables the T cell to receive co-stimulatory signals.

T-lymphocytes are obtained from a patient by leukopheresis, and the appropriate allogenic or autologous T-cell subset (or a mixture of various T-cell subsets), for example, Helper T-cells (T_{H}), cytotoxic T-cells, and Central Memory T-cells (T_{CM}) are genetically altered to express the FLCAR, then administered back to the patient by any clinically acceptable means, to achieve anti-cancer therapy. As noted, T-cells, for example, patient-specific, autologous T-cells engineered to express the FLCARs described herein can be expanded and used in therapy. Various T-cell subsets can be used. In addition, the FLCAR can be expressed in other immune cells such as NK cells. Where a patient is treated with an immune cell expressing an FLCAR described herein, the cell can be an autologous or allogenic T-cell for example. In some cases, the cells used are CD4⁺ and CD8⁺ central memory T-cells (T_{CM}), which are CD45RO₊CD62L⁺, and the use of such cells can improve long-term persistence of the cells after adoptive transfer compared to the use of other types of patient-specific T-cells.

Some embodiments of the present disclosure relate to a pharmaceutical composition comprising humancells which include a nucleic acid sequence encoding an FLCAR including an antigen binding domain (a target-specific binding element), a transmembrane domain, a costimulatory signaling region, and a CD3 zeta signaling domain. The cell may be selected from the group consisting of a T-cell, a natural killer (NK) cell, NK-92 cell, a cytotoxic T-lymphocyte (CTL), and a regulatory T-cell. Some embodiments of the present disclosure relate to a method for stimulating a T-cell-mediated immune response using the cell population encoding the FLCAR. Between the extracellular domain and the transmembrane domain of the FLCAR, there may be incorporated a spacer domain. As used herein, the term “spacer domain” generally means any oligopeptide or polypeptide that functions to link the transmembrane domain to, either the extracellular domain or, the costimulatory signaling domain of the polypeptide chain. A spacer domain may include from 1 to 300 amino acids, e.g., 1 to 50 amino acids, 1 to 25 amino acids, 1 to 10 amino acids, or 1 to 5 amino acids.

The expression of natural or synthetic nucleic acids encoding FLCARs is typically achieved by operably linking a nucleic acid encoding the FLCAR polypeptide or portions thereof to one or more promoters and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

Generally, the cells activated and expanded as described herein may be utilized in the treatment and prevention of diseases that arise in individuals who are immunocompromised. In particular, the engineered cells of the present disclosure are used in the treatment of cancer. In certain embodiments, the cells of the present disclosure are used in the treatment of patients at risk of developing cancer. Thus, the present disclosure provides methods for the treatment or prevention of cancer comprising administering to a subject in need thereof, a therapeutically effective amount of the engineered T-cells of the present disclosure.

The engineered T-cells of the present disclosure may be administered either alone or as a pharmaceutical composition of cells in combination with diluents and/or with other components such as IL-2 or other cytokines or cell populations. Briefly, pharmaceutical compositions of the present disclosure may include an FLCAR-modified cell population as described herein, in combination with one or more pharmaceutically or physiologically acceptable carriers, diluents or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The pharmaceutical compositions of cells of the present disclosure may be administered in a manner appropriate to the disease to be treated (or prevented). Cell compositions of the present disclosure may be formulated for intravenous administration. The quantity and frequency of administration will be determined by such factors as the condition of the patient, and the type and severity of the patient's disease, although appropriate dosages may be determined by clinical trials.

When “an immunologically effective amount”, “an anti-cancer effective amount”, “a cancer-inhibiting effective amount”, or “therapeutic amount” is indicated, the precise amount of the cell compositions of thepresent disclosure to be administered can be determined by a physician with consideration of individual differences in age, weight, tumor size, extent of infection or metastasis, and condition of the patient (subject). In certain embodiments, a pharmaceutical composition comprising the FLCAR T-cells, or other FLCAR-modified cells described herein may be administered at a dosage of, for example, 10⁴ to 10⁹ cells/kg body weight, e.g., 10⁵ to 10⁸ cells/kg body weight, including all integer values within those ranges. T-cell compositions may also be administered multiple times at these dosages. The cells can be administered by using infusion techniques that are commonly known in immunotherapy. An optimal dosage and treatment regimen for a particular patient can readily be determined by one skilled in the art of medicine by monitoring the patient for signs of disease and adjusting the treatment accordingly.

In certain embodiments, it may be desired to administer activated T-cells to a subject and then subsequently redraw blood (or have an apheresis performed), activate T-cells therefrom according to the present disclosure, and reinfuse the patient with these activated and expanded T-cells. This process can be carried out multiple times every few weeks. In certain embodiments, T-cells can be activated from blood draws of from 10 cc to 400 cc. In certain embodiments, T-cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, may select out certain populations of T-cells.

The administration of the disclosed cell compositions may be carried out in any convenient manner, including by aerosol inhalation, injection, ingestion, transfusion, implantation or transplantation. The compositions described herein may be administered to a patient subcutaneously, intradermally, intratumorally, intranodal, intramedullary, intramuscularly, by intravenous (i.v.) injection, by intrathecal route (e.g., for treating a leukemia), or intraperitoneally. In one embodiment, the T-cell compositions of the present disclosure are administered to a patient by intradermal or subcutaneous injection. In another embodiment, the T-cell compositions of the present disclosure are preferably administered by i.v. injection. The compositions of T-cells may be injected directly into a tumor, lymph node, or site of infection.

In certain embodiments of the present disclosure, cells activated and expanded using the methods described herein, or other methods known in the art where T-cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, or monoclonal antibody therapies. In further embodiments, the T-cells of the present disclosure may be used in combination with chemotherapy, radiation, and immunosuppressive agents.

Examples of FL mutant sequences which can be used in the presently disclosed embodiments include, but are not limited to, high affinity isoforms of FL which still maintain a structural resemblance to wild type, and which have binding affinity substantially greater than or equal to the wild type FL, including the L-3H, H8Y, K84E, K84T, W118R, and Q122R mutants, and any combination thereof, where numbering refers to the 156-residue extracellular domain of hFL (see Graddis et al., J. Biol. Chem., Vol. 273, No. 28, pp. 17626-17633, Jul. 10, 1998). The present disclosure is intended to include variants which comprise substitutions by any of the 20 natural amino acids at one or more of said positions, wherein the substituted variant has binding affinity to FLT3 which is substantially equal to or greater than the wild type.

The present disclosure is further described with reference to the following examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teachings provided herein.

### Experimental

Methods

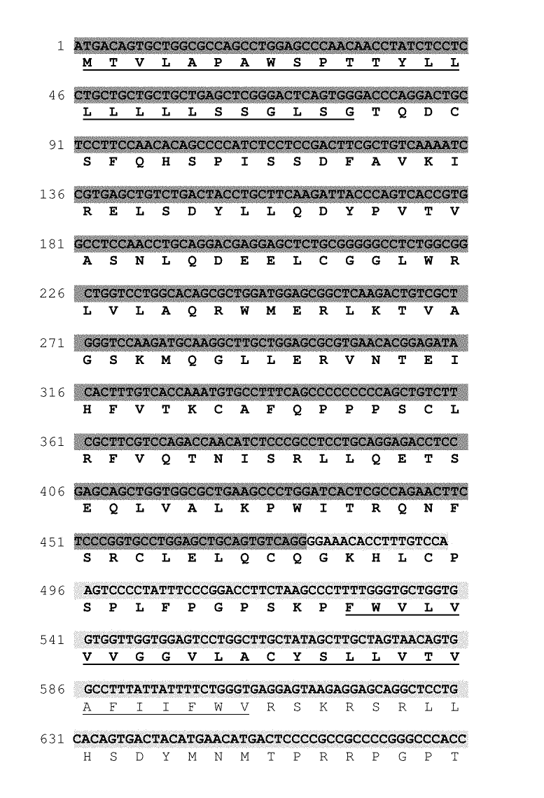

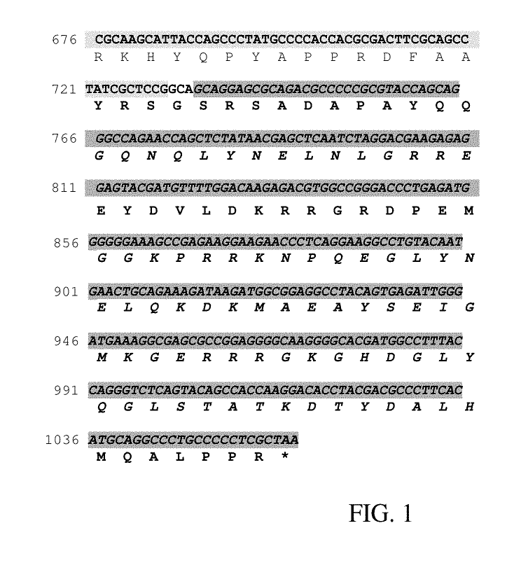

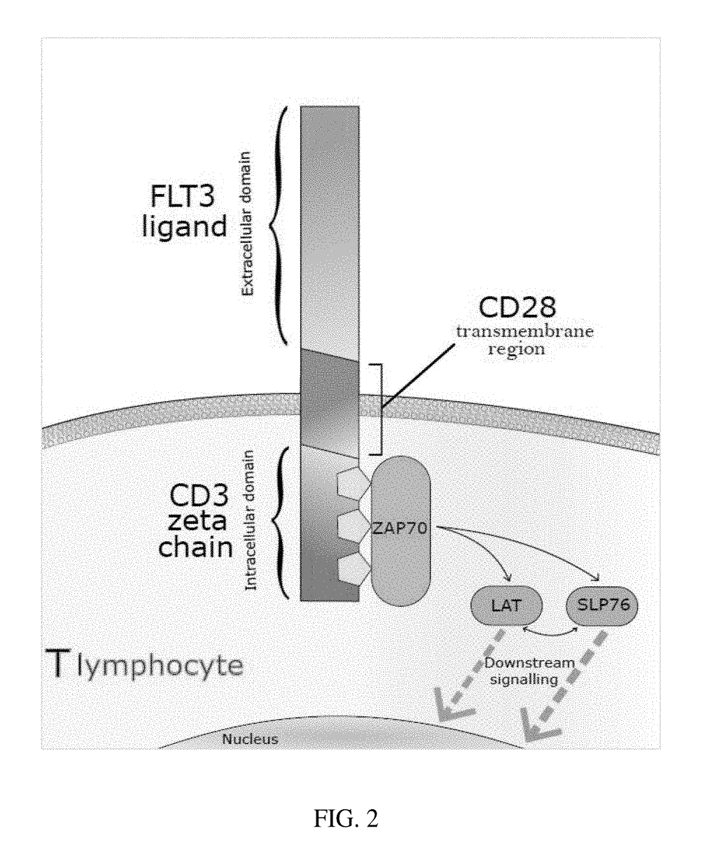

A DNA construct (SEQ ID NO:1) which encodes an FLCAR polypeptide (SEQ ID NO:2) which was used to transfect T-cells to form FLCAR cells is shown in FIG. 1. The DNA construct of FIG. 1 particularly encodes in series a (1) a 159 amino acid portion of FL (SEQ ID NO:3) which comprises, in series, a 26 amino acid FL signal sequence (SEQ ID NO:4) and a 133 amino acid FLT3-binding portion of the FL (SEQ ID NO:5), a first glycine spacer, an 83-amino acid CD28 domain (transmembrane and costimulatory intracellular regions), a second glycine spacer, and a 118-amino acid CD3-zeta intracellular signaling domain comprising a plurality of ITAMs. The 26 amino acid FL signal sequence (SEQ ID NO:4) is cleaved from the protein before the FLCAR becomes functional. The resulting FLCAR construct is shown schematically in FIG. 2.

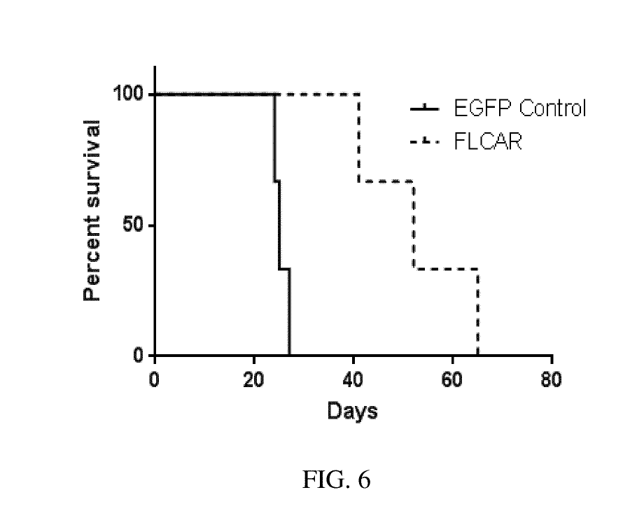

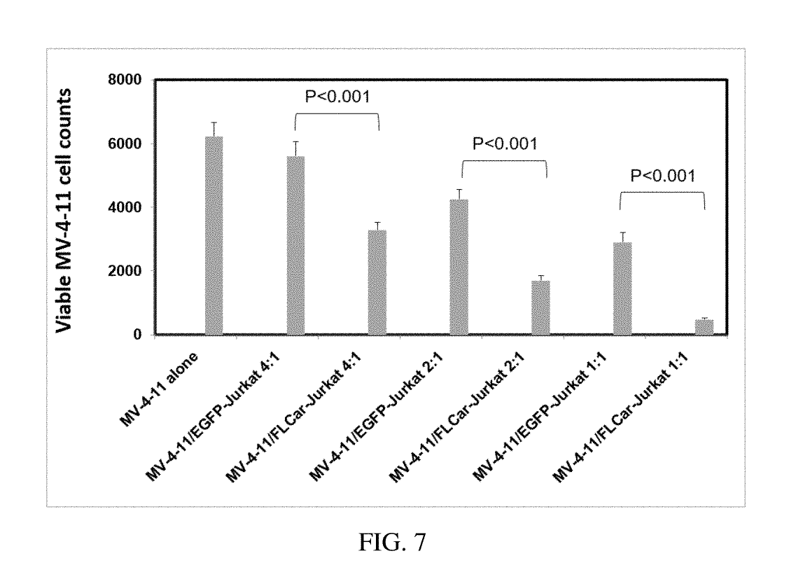

This DNA construct was cloned into the pHIV-EGFP lentiviral vector for expression in T-lymphocyte cell-lines and primary T-cells obtained from healthy donors. An empty pHIV-EGFP vector was used as a negative control. FLCAR was expressed on both CD4⁺ and CD8³⁰ T-lymphocytes, confirmed by western blot. Cell cytoxicity was evaluated by co-culturing FLCAR T-cells and AML cells followed by flow cytometric analyses. Cytokine production was assessed by analyzing expression of interleukin-2 using quantitative RT-PCR. To assess the effects of the FLCAR T-cells in vivo, immuno-deficient NSG-S GM mice were injected through the retro-orbital vein with MV-4-11 cells and primary T-cells expressing EGFP or FLCAR. Survival of treated mice was monitored on a daily basis and is shown in FIGS. 6 and 7.

Results

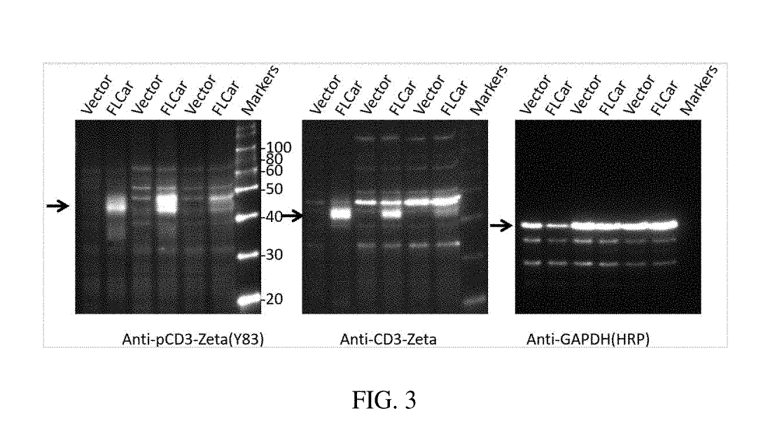

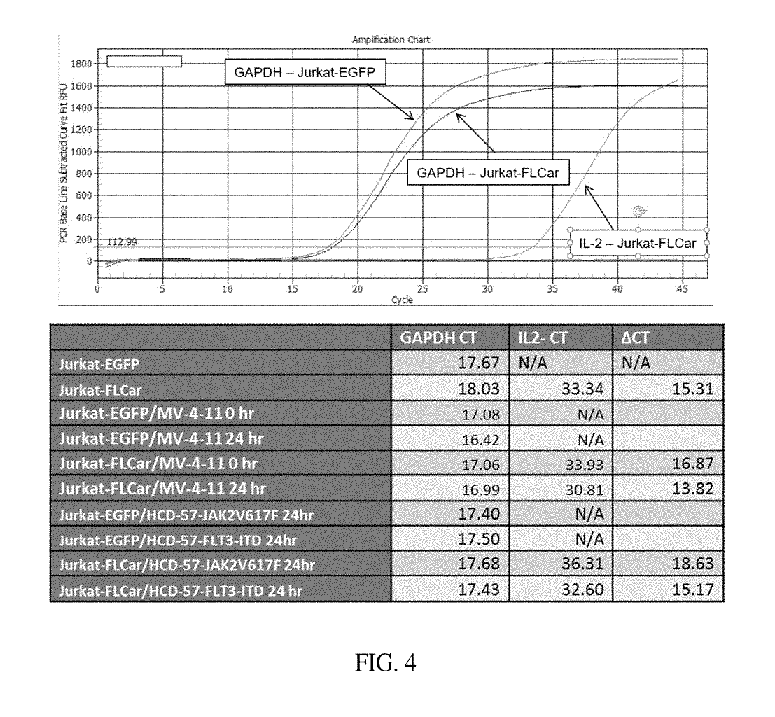

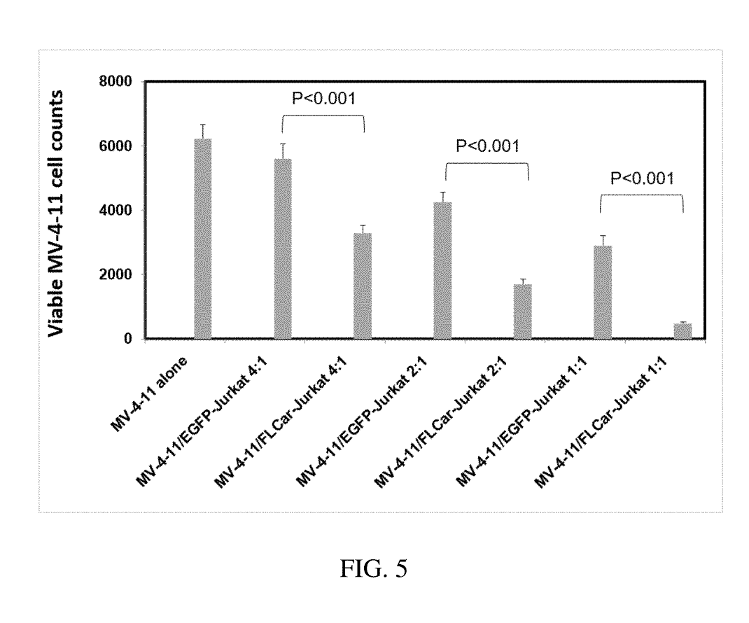

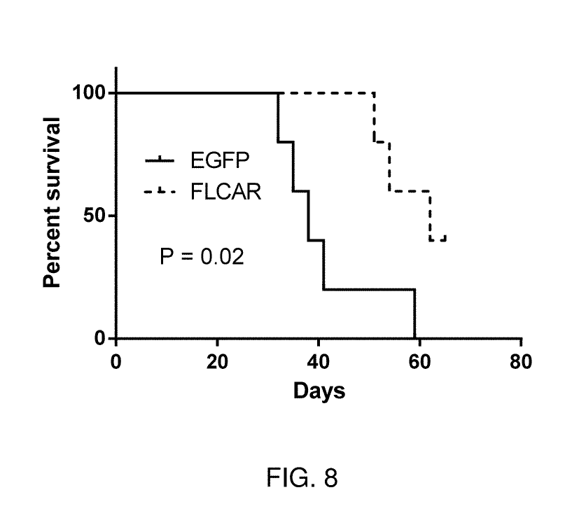

The FLCAR T-cells were generated from CD4⁺ Jurkat and CD8⁺ TK-1 cell lines with up to 80% lentiviral transduction efficiency. The efficiency for primary T-cells was lower (5-20%). FLCAR was expressed as an about 42 kDa protein in cells and was partially phosphorylated on tyrosine (FIG. 3). The expression of FLCAR on lymphocytes lead to increased basal IL-2 expression in the cells. This was further augmented (by >5 fold) upon co-incubating FLCAR T-cells with FLT3-expressing target cells (FIG. 4). Jurkat cells, TK-1 cells and primary human T-cells expressing FLCAR suppressed the growth of FLT3-expressing AML cell lines and primary AML cells in vitro (FIG. 5). Further, FLCAR-expressing primary T-cells significantly prolonged the survival of immune-deficient mice engrafted with FLT3-expressing MV-4-11 leukemia cells (FIGS. 6-7). The results demonstrate that FLCAR can be effectively expressed on T-lymphocytes and mediate potent cytotoxicity against FLT3-expressing AML cells in vitro and in vivo. FIG. 8 shows that administration of FLCAR-T cells prolongs survival of PDX mice engrafted with primary AML cells from patients.

Using FDA approved techniques for cell selection, cloning, and expansion modified CD4⁺ and CD8⁺ CAR T-lymphocyte clones with FLT3-specific cytolytic activity were generated from healthy donor lymphocytes and established T-cell lines in multiple different experiments (FIG. 3). We found significantly higher expression of IL-2 cytokine, which is used as a marker of lymphocyte activation, in FLCAR T-cells as compared to EGFP T-cells (negative control) upon incubation with FLT3-expressing cells (FIG. 4). The experiments have also demonstrated significantly high cytotoxicity of FLCAR T-cells against FLT3-expressing cells as compared to the controls (FIG. 5). FIGS. 6 and 7 show that administration of FLCAR T-cells prolongs survival of mice engrafted with leukemia cells.

The FLCAR of the present disclosure can be expressed on a variety of immunological cell types including, but not limited to, T-lymphocytes, B-lymphocytes, NK cells and macrophages by using any of the existing standard molecular cloning, gene editing, and transduction techniques. Furthermore, the costimulatory signaling domain/trans membrane domain and intracellular signaling domains are not limited to CD28 and CD3-zeta (respectively), but can comprise one or more of the many proteins that are being employed in contemporary immunotherapeutic applications, for example as discussed elsewhere herein. Thus the sequence shown in FIG. 1 is exemplary and is not considered to be limiting of the FLCAR constructs that can be used in embodiments of the present disclosure.

As noted elsewhere herein, the FLCAR T-cells described herein can be used for treatment of patients with AML, MDS, CML in blast crisis, or as a preparative regime for patients needing HSCT. For example, the FLCAR T-cells can be made from lymphocytes obtained from the patients (autologous) or from healthy subjects (allogenic). FLCAR cells can potentially be useful against lymphoblastic leukemias and lymphomas. With appropriate set up and ancillary support it is envisioned that the overall turnaround time from lymphocyte collection to FLCAR T-cell administration will be somewhere between 3-4 weeks.

As evidenced herein, while contemporary adoptive T-cell therapies against AML use monoclonal antibodies against cancer antigens we have employed in certain embodiments portions of the naturally occurring FL protein (or variants thereof) to target FLT3-expressing AML cells by designing chimeric T-lymphocytes designated herein as FLCAR T-cells. However, conventional thinking would consider it counterintuitive to use FL to manufacture a CAR cell against a FLT3-containing cancer cell since FL is a growth factor and thus it would be unexpected by a person of ordinary skill in the art that an agent such as FL, which is known to cause proliferation of AML cells (or cells of myeloid lineage in general), would be effective in turning the body's own lymphocytes against AML cells (or cells of myeloid lineage in general). In spite of this, the present work has confirmed successful transduction of T-cell lines and primary T-cells obtained from donors. We have demonstrated the anti-AML cytotoxic effect ex vivo by direct killing of leukemia cells and enhanced production of cytokines from FLCAR T-cells and in vivo by prolonging survival of mice bearing leukemia cells.

In various aspects, the present disclosure is directed to stably expressing an FL extracellular region on the surface of T-lymphocytes to redirect their cytotoxicity towards FLT3-expressing AML cells and myeloid cells. In one embodiment, the FL extracellular region is combined in series with a costimulatory protein such as CD28 and T-cell receptor CD3 zeta chain to make the final FLCAR construct. Optionally a Kozak sequence (e.g., GCCGAA-SEQ ID NO:28) may be placed in the beginning of FLCAR DNA construct to enhance translation efficiency. A specific extracellular region of the FL protein must be selected such that the signal sequence and FLT3-binding region are intact and included and the protease cleavage (shedding) site and a structurally rigid proline-rich region are excluded. Optionally, the protease cleavage site may be mutated to make it non-subject to cleavage by a protease. A specific part of the costimulatory protein must be selected so that its signal sequence is excluded but its transmembrane and costimulatory domain is retained. A first spacer sequence is optionally inserted between the FL extracellular region and the transmembrane sequence to provide structural flexibility. The spacer may be a single glycine residue (e.g., as shown in the construct in FIG. 1) or a plurality of glycine residues (e.g., from 2 to 10 or more A specific part of the CD3-zeta must be selected so that all of its intracellular tyrosine-based activation motifs are included. Similarly, a second spacer sequence is optionally inserted between the costimulatory domain and the CD3 zeta chain. In non-limiting embodiments, the spacers may be single glycine residues (as shown in the construct in FIG. 1) or a plurality of glycine residues (e.g., from 2 to 10 or more).

As described above, in alternate embodiments, the FLCAR construct may comprise mutant forms of FL which are able to bind with high affinity to FLT3, for example, L-3H, H8Y, K84E, K84T, W118R, and Q122R are examples of such mutation sites. The positions may be substituted with any other amino acid that provides a FL with equivalent or enhanced binding affinity. Alternate signal sequences may be used for more efficient protein processing and membrane distribution of FLCAR. The FL-to-CD28 and CD28-to-CD3-zeta joint region positions may be modified to optimize function of each part of FLCAR. The number of intra-cellular tyrosine-based activation motifs (ITAMs) can be increased and their degree of phosphorylation can be altered to further enhance the activity of FLCAR T-cells. Modifications may include changes to minimize or maximize FL dimerization. The intracellular and transmembrane regions of FLCAR can further be supplemented by contemporary peptide molecules that can add therapeutically desirable properties to the CAR T-cells, for example but not limited to peptides that enable modulation (fine-tuning) of the effector functions of CAR T-cells by providing switch on-off ability (for example, see Rodgers et al., “Switch-mediated activation and retargeting of CAR-T cells for B-cell malignancies,” PNAS, E459-E468, Jan. 12, 2016). For example, in one embodiment, a small switch-off peptide can be added to the intracellular domain that allows quick and easy removal of the FLCAR T-cells from a patient's body if the patient starts having severe side effects due to the FLCAR T-cells.

A non-limiting example of a treatment protocol using the disclosed FLCAR cells includes a clinical scenario in which a newly diagnosed AML or MDS patient undergoes leukocyte apheresis for T-cell collection within the first week of hospital admission. An administration of FLCAR T-cells can be preceded by at least one cycle (each chemo cycle is 3-4 weeks long) of cytoreductive chemotherapy (for instance the conventional regimen of AraC+Daunorubicin may be used). The time for engineering FLCAR T-cells would be about 3-4 weeks from the day of leukocyte apheresis (so FLCAR T-cells will be ready by the time the patient finishes the first cycle of chemo). FLCAR T-cells will be infused at a sufficiently high dose (e.g., 1×10⁶ to 1×10⁷ cells/kg). This should be a slow infusion under close monitoring. Flow cytometry-based assessment of bone marrow should be planned about 3-4 weeks after FLCAR T-cell administration which will determine the disease status. Patients not in remission at that time can be considered for another course of FLCAR T-cells (e.g., same dose or slightly higher dose if the first course was well tolerated) while those in remission can be taken to hematopoietic stem cell transplantation (either autologous or allogenic).

In non-limiting embodiments, the present disclosure is directed to an immunologic cell comprising a chimeric antigen receptor (CAR) molecule comprising an FLT3-binding portion of Fms-related tyrosine kinase 3 ligand (FL). The immunogenic cell may be any immunological cell, such as a T-lymphocyte, B-lymphocyte, NK cell, or macrophage for example. The FLCAR further comprises a transmembrane portion, at least one intracellular costimulatory domain, such as but not limited to, all or portions of a CD28 costimulatory domain, and an intracellular signaling domain, e.g., a CD3-zeta chain. Other costimulatory domains which may be used instead include, but are not limited to, those from CD27, 4-1BB (CD137), and OX40 (CD134). In at least certain embodiments, the FL extracellular region of the FLCAR comprises a signal peptide (prior to removal thereof), terminates prior to the transmembrane region, includes the full FLT3-binding region, and excludes the FL cleavage site. The FLCAR cells may be used in a therapy for treating any disease involving a cell which expresses FLT3, and/or as a part of myeloablative therapy prior to HSCT, including but not limited to severe combined immune deficiency (SCID) and enzyme disorders including but not limited to Hurler syndrome.

The disclosure is not limited to the FLCAR-encoding construct shown in Table 1 (FIG. 1, i.e., SEQ ID NO:1), or to the protein thereby encoded as shown in Table 2 (SEQ ID NO:2).

TABLE 1
FLCAR construct DNA sequence (SEQ ID NO: 1)

ATGACAGTGCTGGCGCCAGCCTGGAGCCCAACAACCTATCTCCTCCTGCT
GCTGCTGCTGAGCTCGGGACTCAGTGGGACCCAGGACTGCTCCTTCCAAC
ACAGCCCCATCTCCTCCGACTTCGCTGTCAAAATCCGTGAGCTGTCTGAC
TACCTGCTTCAAGATTACCCAGTCACCGTGGCCTCCAACCTGCAGGACGA
GGAGCTCTGCGGGGGCCTCTGGCGGCTGGTCCTGGCACAGCGCTGGATGG
AGCGGCTCAAGACTGTCGCTGGGTCCAAGATGCAAGGCTTGCTGGAGCGC
GTGAACACGGAGATACACTTTGTCACCAAATGTGCCTTTCAGCCCCCCCC
CAGCTGTCTTCGCTTCGTCCAGACCAACATCTCCCGCCTCCTGCAGGAGA
CCTCCGAGCAGCTGGTGGCGCTGAAGCCCTGGATCACTCGCCAGAACTTC
TCCCGGTGCCTGGAGCTGCAGTGTCAGGGGAAACACCTTTGTCCAAGTCC
CCTATTTCCCGGACCTTCTAAGCCCTTTTGGGTGCTGGTGGTGGTTGGTG
GAGTCCTGGCTTGCTATAGCTTGCTAGTAACAGTGGCCTTTATTATTTTC
TGGGTGAGGAGTAAGAGGAGCAGGCTCCTGCACAGTGACTACATGAACAT
GACTCCCCGCCGCCCCGGGCCCACCCGCAAGCATTACCAGCCCTATGCCC
CACCACGCGACTTCGCAGCCTATCGCTCCGGCAGCAGGAGCGCAGACGCC
CCCGCGTACCAGCAGGGCCAGAACCAGCTCTATAACGAGCTCAATCTAGG
ACGAAGAGAGGAGTACGATGTTTTGGACAAGAGACGTGGCCGGGACCCTG
AGATGGGGGGAAAGCCGAGAAGGAAGAACCCTCAGGAAGGCCTGTACAAT
GAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCA
GTACAGCCACCAAGGACACCTACGACGCCCTTCACATGCAGGCCCTGCCC
CCTCGCTAA

TABLE 2
FLCAR construct protein sequence (SEQ ID NO: 2)

MTVLAPAWSPTTYLLLLLLLSSGLSGTQDCSFQHSPISSDFAVKIRELSD
YLLQDYPVTVASNLQDEELCGGLWRLVLAQRWMERLKTVAGSKMQGLLER
VNTEIHFVTKCAFQPPPSCLRFVQTNISRLLQETSEQLVALKPWITRQNF
SRCLELQCQGKHLCPSPLFPGPSKPFWVLVVVGGVLACYSLLVTVAFIIF
WVRSKRSRLLHSDYMNMTPRRPGPTRKHYQPYAPPRDFAAYRSGSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR

In alternate embodiments, for example as shown in Table 3, the FL extracellular region of the construct may comprise fewer than the 133 amino acid residues of SEQ ID NO:5 , as long as the shorter FL extracellular region comprises a domain that binds to FLT3 (i.e., is an FLT3-binding domain) with substantially equal or greater affinity as the wild type 133-amino acid sequence of SEQ ID NO:5. For example, a shorter FL extracellular region may be a portion of SEQ ID NO:5 comprising or consisting of as few as 100 amino acids, e.g., starting at position 8 of SEQ ID NO:5 and ending at position 117 of SEQ ID NO:5, i.e., a protein having the amino acid sequence SEQ ID NO:6. In alternate embodiments, the FL extracellular region may comprise any portion of SEQ ID NO:5 as long as it comprises the 100 contiguous amino acids of SEQ ID NO:6, or a variant sequence having at least 90% identity (as defined elsewhere herein) to SEQ ID NO:6, and binds to FLT3 with substantially equal or greater affinity as the wild type 133-amino acid sequence of SEQ ID NO:5. In certain embodiments, the FL extracellular region is a sequence comprising SEQ ID NO:6 and which is further extended from the N-terminal end with a sequence of 1, 2, 3, 4, 5, 6, or 7 amino acids (such as but not limited to Q, FQ, SFQ, CSFQ (SEQ ID NO:7), DCSFQ (SEQ ID NO:8), QDCSFQ (SEQ ID NO:9), or TQDCSFQ (SEQ ID NO:10), respectively). In certain embodiments, the extracellular FLT3-binding domain is a sequence comprising SEQ ID NO:6 and which is further extended from the C-terminal end with a sequence of 1, 2, 3, 4, 5, 6, 7, 8, 9, 10, 11, 12 13, 14, 15, or 16 amino acids (such as but not limited to, W, WI, WIT, WITR (SEQ ID NO:11), WITRQ (SEQ ID NO:12), WITRQN (SEQ ID NO:13), WITRQNF (SEQ ID NO:14), WITRQNFS (SEQ ID NO:15), WITRQNFSR (SEQ ID NO:16), WITRQNFSRC (SEQ ID NO:17), WITRQNFSRCL (SEQ ID NO:18), WITRQNFSRCLE (SEQ ID NO:19), WITRQNFSRCLEL (SEQ ID NO:20), WITRQNFSRCLELQ (SEQ ID NO:21), WITRQNFSRCLELQC (SEQ ID NO:22), or WITRQNFSRCLELQCQ (SEQ ID NO:23), respectively). The sequence comprising SEQ ID NO:6 may be extended in both directions. In alternate embodiments, the FL extracellular region may comprise a variant of SEQ ID NO:5 or SEQ ID NO:6, or other sequences described above, where the variant sequence binds to FLT3 with substantially equal or greater affinity as the wild type SEQ ID NO:5 and has at least 90% identity to the corresponding SEQ ID NO:5, SEQ ID NO:6, or other sequence described herein.

TABLE 3
FL subsequences

SEQ ID NO: 3 (amino acids 1-159)

| | |
|---|---|
| 1 | MTVLAPAWSP TTYLLLLLLL SSGLSGTQDC SFQHSPISSD FAVKIRELSD |
| 51 | YLLQDYPVTV ASNLQDEELC GGLWRVLAQ RWMERLKTVA GSKMQGLLER |
| 101 | VNTEIHFVTK CAFQPPPSCL RFVQTNISRL LQETSEQLVA LKPWITRQNF |
| 151 | SRCLELQCQ |

SEQ ID NO: 4 (amino acids 1-26)

| | |
|---|---|
| 1 | MTVLAPAWSP TTYLLLLLLL SSGLSG |

SEQ ID NO: 5 (amino acids 27-159)

| | |
|---|---|
| 27 | TQDCSFQHSP ISSDFAVKIR ELSDYLLQDY PVTVASNLQD EELCGGLWRL |
| 77 | VLAQRWMERL KTVAGSKMQG LLERVNTEIH FVTKCAFQPP PSCLRFVQTN |
| 127 | ISRLLQETSE QLVALKPWIT RQNFSRCLEL QCQ |

SEQ ID NO: 6 (amino acids 34-143)

| | |
|---|---|
| 34 | HSPISSDFAV KIRELSDYLL QDYPVTVASN LQDEELCGGL WRLVLAQRWM |
| 84 | ERLKTVAGSK MQGLLERVNT EIHFVTKCAF QPPPSCLRFV QTNISRLLQE |
| 134 | TSEQLVALKP |

It will be understood from the foregoing description that various modifications and changes may be made in the various embodiments of the present disclosure without departing from their true spirit. The description provided herein is intended for purposes of illustration only and is not intended to be construed in a limiting sense. Thus, while the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts. Changes may be made in the formulation of the various components, constructs, cells and compositions described herein, the methods described herein or in the steps or the sequence of steps of the methods described herein without departing from the spirit and scope of the present disclosure.

What is claimed is:

1. A nucleic acid sequence encoding a chimeric antigen receptor (FLCAR) comprising: (1) an extracellular portion of Fms-related tyrosine kinase 3 ligand (FL) that binds to Fms-related tyrosine kinase 3 (FLT3), (2) a transmembrane domain, (3) a costimulatory signaling domain, and (4) an intracellular signaling domain.

2. The nucleic acid sequence of claim 1, wherein the extracellular portion of FL comprises the amino acid sequence of SEQ ID NO:6, or a variant thereof having at least 90% identity to SEQ ID NO:6.

3. The nucleic acid sequence of claim 2, wherein the extracellular portion of FL further comprises 1 to 7 additional amino acids extending from the N-terminal end, and/or 1 to 16 additional amino acids extending from the C-terminal end of SEQ ID NO:6 or the variant thereof.

4. The nucleic acid sequence of claim 1, wherein the extracellular portion of FL comprises the amino acid sequence of SEQ ID NO:5, or a variant thereof having at least 90% identity to SEQ ID NO:5.

5. The nucleic acid sequence of claim 1, wherein the costimulatory signaling domain is derived from the group consisting of CD27, CD28,4-1BB, OX40, CD30, CD40, PD-1, ICOS, lymphocyte function-associated antigen-1 (LFA-1), CD2, CD7, LIGHT, NKG2C, B7-H3, and any combination thereof.

6. The nucleic acid sequence of claim 1, wherein the intracellular signaling domain comprises a CD3 zeta chain.

7. The nucleic acid sequence of claim 1, wherein the extracellular portion of FL is separated from the transmembrane domain via a first spacer peptide, and the costimulatory signaling domain is separated from the intracellular signaling domain via a second spacer peptide.

8. The nucleic acid sequence of claim 1 disposed in a vector.

9. A chimeric antigen receptor (FLCAR), comprising the polypeptide encoded by the nucleic acid sequence of claim 1.

10. An isolated cell, comprising the nucleic acid sequence of claim 1.

11. The isolated cell of claim 10, where the isolated cell is a T-cell.

12. An isolated cell, comprising the chimeric antigen receptor (FLCAR) encoded by the nucleic acid sequence of claim 1.

13. The isolated cell of claim 12, where the isolated cell is a T-cell.

14. A method of stimulating in a subject an immune response against cells which express Fms-related tyrosine kinase 3 (FLT3), comprising: administrating to a subject in need of such therapy an effective amount of a composition of the T-cells of claim 13.

15. The method of claim 14, wherein the subject has acute myeloid leukemia (AML), myelodysplastic syndromes (MDS), chronic myeloid leukemia (CML) in blast crisis, or is in need of a myeloablative treatment prior to hematopoietic stem cell transplantation (HSCT).


SEQUENCE LISTING (28 sequences)

SEQ ID NO: 1 - DNA, 1059 bp, Artificial Sequence
  Completely synthesized in the lab.
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcaggggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 2 - protein, 352 aa, Artificial Sequence
  Completely synthesized in the lab.
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQGKHLCPSPLFPGPSKPFWVLVVVGGVLACYSLLVTVAFIIFWVRSKRSRLLHSDYMNMTPRRPGPTRKHYQPYAPPRDFAAYRSGSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 158 aa, Artificial Sequence
  Completely synthesized in the lab.
MTVLAPAWSPTTYLLLLLLLSSGLSGTQDCSFQHSPISSDFAVKIRELSDYLLQDYPVTVASNLQDEELCGGLWRVLAQRWMERLKTVAGSKMQGLLERVNTEIHFVTKCAFQPPPSCLRFVQTNISRLLQETSEQLVALKPWITRQNFSRCLELQCQ

SEQ ID NO: 4 - protein, 26 aa, Artificial Sequence
  Completely synthesized in the lab.
MTVLAPAWSPTTYLLLLLLLSSGLSG

SEQ ID NO: 5 - protein, 133 aa, Artificial Sequence
  Completely synthesized in the lab.
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQ

SEQ ID NO: 6 - protein, 110 aa, Artificial Sequence
  Completely synthesized in the lab.
HSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKP

SEQ ID NO: 7 - protein, 4 aa, Artificial Sequence
  Completely synthesized in the lab.
CSFQ

SEQ ID NO: 8 - protein, 5 aa, Artificial Sequence
  Completely synthesized in the lab.
DCSFQ

SEQ ID NO: 9 - protein, 6 aa, Artificial Sequence
  Completely synthesized in the lab.
QDCSFQ

SEQ ID NO: 10 - protein, 7 aa, Artificial Sequence
  Completely synthesized in the lab.
TQDCSFQ

SEQ ID NO: 11 - protein, 4 aa, Artificial Sequence
  Completely synthesized in the lab.
WITR

SEQ ID NO: 12 - protein, 5 aa, Artificial Sequence
  Completely synthesized in the lab.
WITRQ

SEQ ID NO: 13 - protein, 6 aa, Artificial Sequence
  Completely synthesized in the lab.
WITRQN

SEQ ID NO: 14 - protein, 7 aa, Artificial Sequence
  Completely synthesized in the lab.
WITRQNF

SEQ ID NO: 15 - protein, 8 aa, Artificial Sequence
  Completely synthesized in the lab.
WITRQNFS

SEQ ID NO: 16 - protein, 9 aa, Artificial Sequence
  Completely synthesized in the lab.
WITRQNFSR

SEQ ID NO: 17 - protein, 10 aa, Artificial Sequence
  Completely synthesized in the lab.
WITRQNFSRC

SEQ ID NO: 18 - protein, 11 aa, Artificial Sequence
  Completely synthesized in the lab.
WITRQNFSRCL

SEQ ID NO: 19 - protein, 12 aa, Artificial Sequence
  Completely synthesized in the lab.
WITRQNFSRCLE

SEQ ID NO: 20 - protein, 13 aa, Artificial Sequence
  Completely synthesized in the lab.
WITRQNFSRCLEL

SEQ ID NO: 21 - protein, 14 aa, Artificial Sequence
  Completely synthesized in the lab.
WITRQNFSRCLELQ

SEQ ID NO: 22 - protein, 15 aa, Artificial Sequence
  Completely synthesized in the lab.
WITRQNFSRCLELQC

SEQ ID NO: 23 - protein, 16 aa, Artificial Sequence
  Completely synthesized in the lab.
WITRQNFSRCLELQCQ

SEQ ID NO: 24 - DNA, 24 bp, Artificial Sequence
  Completely synthesized in the lab.
gggacttaatcagcaatatcaacg

SEQ ID NO: 25 - DNA, 24 bp, Artificial Sequence
  Completely synthesized in the lab.
ctacaatggttgctgtctcatcag

SEQ ID NO: 26 - DNA, 23 bp, Artificial Sequence
  Completely synthesized in the lab.
agggctgcttttaactctggtaa

SEQ ID NO: 27 - DNA, 23 bp, Artificial Sequence
  Completely synthesized in the lab.
tgggtggaatcatattggaacat

SEQ ID NO: 28 - DNA, 6 bp, Artificial Sequence
  Completely synthesized in lab.
gccgaa